(19) United States
(12) Patent Application Publication
(10) Pub. No.: US 2025/0364075 A1
(43) Pub. Date: Nov. 27, 2025

(54) COMPUTATIONAL METHODS TO IDENTIFY ALLOSTERIC SITES THAT MODULATE ENZYME ACTIVITY

(71) Applicants: Ismail Can Kazan, Tempe, AZ (US); Jeremy Mills, Tempe, AZ (US); Sefika Banu Ozkan, Phoenix, AZ (US)

(72) Inventors: Ismail Can Kazan, Tempe, AZ (US); Jeremy Mills, Tempe, AZ (US); Sefika Banu Ozkan, Phoenix, AZ (US)

(21) Appl. No.: 19/215,650

(22) Filed: May 22, 2025

Related U.S. Application Data
(60) Provisional application No. 63/650,607, filed on May 22, 2024.

(51) Int. Cl. G16B 15/30; C12N 9/06; G16B 5/00

(52) U.S. Cl. CPC G16B 15/30; C12N 9/003; G16B 5/00; C12Y 105/01003

(57) ABSTRACT

A method of characterizing a protein is provided herein. The method includes accessing simulated protein structure data with a computer system, where the simulated protein structure data indicate a structure of the protein. The method further includes quantifying, using the computer system and based on the simulated protein structure data, a plurality of dynamics metrics for a plurality of residues in the protein. The plurality of dynamics metrics are related to functional behaviors of the protein using the computer system. Additionally, the method includes generating a report from the functional behaviors and the dynamics metrics using the computer system, where the report comprises a functional characterization of each residue in the plurality of residues and a functional characterization of the protein.

### CROSS-REFERENCE TO RELATED APPLICATIONS

This application claims the benefit of U.S. Provisional Patent Application Ser. No. 63/650,607, filed on May 22, 2024, and entitled “COMPUTATIONAL METHODS TO IDENTIFY ALLOSTERIC SITES THAT MODULATE ENZYME ACTIVITY,” which is herein incorporated by reference in its entirety.

### STATEMENT OF FEDERALLY SPONSORED RESEARCH

This invention was made with government support under 1901709 awarded by National Science Foundation. The government has certain rights in the invention.

### BACKGROUND

There is a need to understand the relationship between protein sequence-structure and function. One area of interest is using deep sequencing to assess the impact of mutations on protein function. However, this is particularly challenging for complex enzymes. It has been demonstrated that mutations in regions distal to active sites can impact protein function. Therefore, there is a need to characterize protein dynamics to improve the ability to predict protein behavior and predict the impact of protein mutations.

### SUMMARY OF THE DISCLOSURE

According to an aspect of the present disclosure, a method of characterizing an allosteric site of a protein is provided. The method includes accessing simulated protein structure data with a computer system, wherein the simulated protein structure data indicate a structure of the protein. The method also includes quantifying, using the computer system and based on the simulated protein structure data, a plurality of dynamics metrics for a plurality of residues in the protein. The method further includes relating the plurality of dynamics metrics to functional behaviors of the protein using the computer system. Additionally, the method includes generating a report from the functional behaviors and the dynamics metrics using the computer system, wherein the report includes a functional characterization of each residue in the plurality of residues and a functional characterization of the allosteric site of the protein.

According to other aspects of the present disclosure, the method may include one or more of the following features. The protein structure data may be simulated using molecular dynamics. The plurality of dynamics metrics may include at least one of a dynamic flexibility index, a dynamic coupling index, or an asymmetric dynamic coupling index. The plurality of dynamics metrics may include a solvent accessible surface area. The protein may include a drug target. The drug target may include dihydrofolate reductase (DHFR). The functional characterization of the protein may include indicating long-range coupling dynamics. The long-range coupling dynamics may include identifying controller residues or regions and controlled residues or regions. The report may further include a functional characterization of one or more domains in the protein. The report may indicate a prediction of an impact of one or more mutations to the protein based on the functional characterization of the protein and the dynamics metrics. The report may indicate a prediction of an impact of one or more drugs on the protein function based on the functional characterization of the protein and the dynamics metrics.

According to another aspect of the present disclosure, a method of identifying allosteric sites in a protein is provided. The method includes simulating protein structure data using a computer system, wherein the protein structure data indicate a structure of the protein. The method also includes calculating an asymmetric dynamic coupling index (DCI_{asym}) for a plurality of residues in the protein using the computer system. The method further includes classifying residues as controller or controlled based on their DCI_{asym} values. Additionally, the method includes identifying allosteric sites in the protein based on the classification of residues as controller or controlled. The method also includes generating a report that indicates the allosteric sites in the protein.

According to other aspects of the present disclosure, the method may include one or more of the following features. Classifying residues as controller or controlled may include classifying residues with DCI_{asym} values above an upper threshold as controlled residues and classifying residues with DCI_{asym} values below a lower threshold as controller residues. The upper threshold may be 0.05 and the lower threshold may be −0.05. The method may further include calculating a dynamic flexibility index (DFI) for each residue in the protein. Identifying the allosteric sites may include identifying controller residues with high DFI values as potential allosteric sites. Calculating the DCI_{asym} for each of the plurality of resides may include calculating a dynamic coupling index (DCIᵢⱼ) for each of the plurality of residues, wherein the DCI of a residue position i indicates its response to a perturbation on another reside position j, and calculating the DCI_{asym} values as a magnitude of difference between dynamic coupling scores of residue positions i to j (DCIᵢⱼ) versus dynamic coupling scores of residue positions j to i (DCIⱼᵢ). The method may further include predicting an impact of mutations on protein function at the identified allosteric sites. Predicting the impact of mutations may include classifying potential mutations as beneficial, neutral, or deleterious to protein function based on at least the DCI_{asym} values. The generated report may further indicate a functional characterization of the identified allosteric sites and predictions for the impact of mutations at those sites.

### BRIEF DESCRIPTION OF THE DRAWINGS

The features and advantages of the present disclosure, and the manner of attaining them, will become more apparent and the present disclosure will be better understood by reference to the description of the present disclosure taken in conjunction with the accompanying drawings, wherein:

FIG. 1: Example of a process for generating a report that indicates a functional characterization of a protein in accordance with some embodiments of the disclosed subject matter.

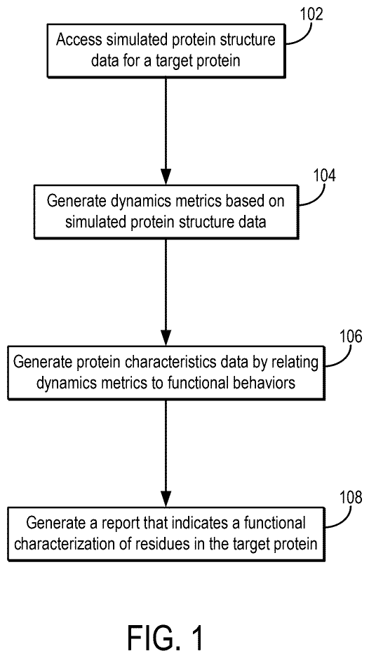

FIGS. 2A-2C: (FIG. 2A) DFI profile of DHFR projected on the crystal structure with PDB ID: 1rx2. Regions of high flexibility are colored red; regions of medium flexibility are colored white and highly rigid regions are colored blue. (FIG. 2B) Functionally critical residues in the M20 (residues 8-24) and FG (residues 116-132) loops are shown as spheres colored by their DFI scores. (FIG. 2C) The DFI profile of apo DHFR. D122 and T123 in the FG loop; and V13, G15, N18, and A19 on the M20 loop are highlighted with red colored vertical lines.

FIGS. 3A-3B: DFI score distributions for the five previously defined functional classes with and without in the presence of Lon protease. (FIG. 3A) In the absence of Lon protease the “Intolerant” labeled residues almost always display very low DFI values, followed by the residues labeled as “Restricted” showing an overall rigid behavior (i.e., % DFI<0.6). Conversely, “Beneficial” and “Tolerant” residues are more commonly found in high DFI regions of the protein (i.e., % DFI≥0.6). The differences in these distributions are statistically significant, with p-values 0.0002 and 2e-05 respectively, calculated by Fisher's exact test using 0.6% DFI as the threshold value. Residues labeled “Mixed” are distributed across different DFI ranges. (FIG. 3B) In the presence of Lon protease, DFI scores of the residues distributed among functional classes are similar to those when the Lon protease is absent.

FIGS. 4A-4D: An analysis of DHFR using DCI_{asym}. FIG. 4A and FIG. 4C show DCI profiles measuring the dynamic coupling of the M20 (FIG. 4A), and FG (FIG. 4C) loops projected onto the DHFR structure; high coupling is shown in purple, low coupling is shown in green. FIG. 4B and FIG. 4D show the distribution of DCI_{asym} values of all residues calculated by targeting the M20 (FIG. 4B) and FG (FIG. 4D) loops. Positive DCI_{asym} values indicate that the residues within the loop control interactions with other residues while negative values represent residues that control the dynamics of the loop.

FIG. 5: Analyses of the “controller” and “controlled” classified average selection coefficient value distributions (+Lon) for the M20 and FG loops. For both M20 and FG loops the average selection coefficient value distributions are different for “controller” and “controlled” labels. The residues with “controller” labels are commonly distributed near either neutral/enhanced (near zero, or positive) region while “controlled” residues display a distribution among negative values (deleterious; M20 loop: p=0.005, and FG loop: p=0.008, Student's t-test). The gray distribution (line as the mean and shade as the variance) is generated by randomly selecting a different subset of residues (excluding “controller”/“controlled” residues) five times. Comparison of the randomly selected positions' average selection coefficient distributions with those of “controller” and “controlled,” distributions of both M20 loop and FG loop shows that randomly selecting residues fails to capture the selection coefficient distribution of the “controller” residues (average p values over five random samples are 0.028, and 0.001, respectively) and “controlled” residues (p<0.043 and <0.0425, respectively).

FIGS. 6A-6B: Experimentally measured selection coefficient values of “controller” and “controlled” residues of the M20 and FG loops. (FIG. 6A) A violin plot of average selection coefficient values of the residues controlling both loops suggests that these residues have a peak on positive values compared to those residues that are controlled by the loops (p=3e-7). This suggests that mutations to residues that are controller of the M20, and FG loops can potentially enhance the activity of DHFR, while mutations to residues controlled by these loops are mostly deleterious. (FIG. 6B) A violin plot generated using the selection coefficient of all amino acid substitutions per position. The distribution of selection coefficient values for “controller” residues falls primarily in the neutral to positive range. Alternatively, a broader distribution is observed for residues controlled by both loops; mutations at these residues often have a drastic negative impact on activity.

FIG. 7: Conservation distribution of DHFR positions designated either controlled by or controllers of the M20 and FG loops. Conservation values are obtained using ConSurf database (Ben Chorin et al., 2020; Goldenberg et al., 2009). Residues that are “controller” attain lower values (nonconserved) compared to “controlled” residues which are more distributed on higher (conserved) values. The Student's t-test showed that the difference in distribution was statistically significant (p=0.003).

FIG. 8: Example of a system for generating a report that indicates a functional characterization of a protein in accordance with some embodiments of the disclosed subject matter.

FIG. 9: Box plot of DFI values for two sets of residues related to their protease sensitivity. Residues that are tolerant to Lon protease have slightly lower DFI scores compared to the one that are susceptible (p<0.23). This shows that the susceptible residues have a higher degree of flexibility than the tolerant residues. This observation is interesting because it may suggest that the degree of flexibility of a residue play a role in its susceptibility to protease activity and its overall stability.

FIGS. 10A-10B: The asymmetry labeled average selection coefficient value (in the absence of Lon) distributions for the M20 and FG loops. (FIG. 10A) Distribution for M20 loop plot shows “controller” and “controlled” labeled distributions are different (p=0.005, Student's t-test). (FIG. 10B) FG loop value distribution show “controller” and “controlled” labeled distributions are different. (p=0.008, Student's t-test).

FIGS. 11A-11B: Violin plots of experimentally measured selection coefficient values of “controller” and “controlled” residues of the GH loop (FIG. 11A) and Adenosine Binding Domain (FIG. 11B). The distribution of selection coefficient values for “controller” residues follows an overall neutral trend. On the other hand, “controlled” residues show a diverse distribution spreading to negative (deleterious) ranges. The difference observed in the distributions of “controlled” and “controller” are statistically significant, with p values 0.003 and 3e-7, for GH loop and Adenosine Binding Domain, respectively.

FIGS. 12A-12G: Correlation plots of binned structural and dynamic features with average selection coefficients (FIG. 12A) Structural features SASA and (FIG. 12B) average number (#) of contacts are binned and compared with experimental data. Both SASA and average # of contacts values of residues correlate well with the experimental data. Structural metrics betweenness, closeness, and eigenvector centrality are compared with average selection coefficient. (FIG. 12C) Betweenness metric binned every 0.1 range shows that ranges from zero to 0.2 and 0.9 to 1.0 have higher fitness values relative to others (R=−0.65). (FIG. 12D) Eigenvector centrality metric is binned every 0.1 range. The eigenvector centrality metric overall shows a good correlation, but all the bins have an experimental value lower than the neutral range (R=−0.85). (FIG. 12E) Closeness metric binned every 0.01 range shows that residues with values from 0.15 to 0.19 shows great promise in enhancing the activity (R=−0.87). (FIG. 12F) M20 loop and (FIG. 12G) FG loop DCI_{asym} value binned every 0.2 window shows that DCI_{asym} values lower than zero yield higher activity compared to those positions in the positive ranged bins. This correlation fits well with the definitions of “controlled” and “controller”.

FIG. 13: A box plot showing % DFI distribution of “controlled” and “controller” residues. These distributions show that “controller” sites attain high % DFI values on average; conversely “controlled” positions are generally found to be rigid.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

In one aspect, the disclosure provides methods and systems of generating a report that quantitatively analyzes a protein or protein structure and provides a functional characterization of the protein. The method may include simulating the structure of a protein using a computer system. The method may include quantifying multiple dynamics metrics for a plurality of residues in the protein. The method may include relating the multiple dynamics metrics to functional behaviors of the protein. The method may further include generating a report using the functional behaviors and the dynamics metrics to provide a functional characterization of each residue in the protein and a functional characterization of the whole protein.

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the disclosure and does not pose a limitation on the scope of the disclosure unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, or example, by a mutated residue. A protein, peptide, or polypeptide may also be a single molecule or may be a multi-molecular complex. A protein, peptide, or polypeptide may also be a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain.

“Functional behavior” of a protein refers to protein activity such as flexibility, rigidity, binding dynamics, or coupling dynamics.

“Functional characterization” of a protein or protein residue refers to a description that relates protein structure to protein behavior. For instance, a functional characterization can provide an explanation of coupling dynamics between protein residues and/or protein domains. A functional characterization can also predict the impact of mutations to residues or protein domains; these predictions can characterize a potential mutation as beneficial (e.g., improves the function or enzymatic activity of a protein) or as deleterious (e.g., inhibits the function or enzymatic activity of a protein). A potential mutation can also be predicted to be neutral and have no significant effect on protein activity. Functional characterization can provide detailed explanations of coupling dynamics. For instance, a functional characterization can define a protein residue or domain as a “controller” (e.g., the residue dynamically controls another region) or “controlled” (e.g., the residue is dynamically controlled by another region). A functional characterization may predict the impact of mutations in controller/controlled regions. A functional characterization can also relate the characterization of a protein to the evolutionary conservation of a protein.

The present disclosure relates to systems and methods for characterizing proteins using computational analysis of protein structure and dynamics. These systems and methods may provide insights into protein function, behavior, and potential responses to modifications or interactions. The approaches described in the present disclosure may have applications in fields such as drug discovery, protein engineering, and understanding disease mechanisms. By providing quantitative analyses of protein dynamics and relating this to function, the disclosed methods may enable new understanding of protein behavior and may make predictions about effects of mutations or drug interactions.

In some cases, the methods described herein may involve simulating protein structures and analyzing various dynamics metrics to assess functional behaviors. The analysis may generate detailed reports characterizing individual residues as well as overall protein function. In some implementations, the methods may leverage computational power to process complex protein structural data and extract meaningful functional insights. This may allow for rapid and comprehensive analysis of proteins that would be challenging or impossible through experimental methods alone.

In accordance with some embodiments of the disclosed subject matter, mechanisms (which can include, for example, systems and methods) for characterizing a protein are provided. Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

Referring now to FIG., a flowchart is illustrated as setting forth the steps of an example method for characterizing a protein based on computational analysis of protein structure and dynamics.

The method may involve accessing simulated protein structure data with a computer system, as indicated at step 02. In some cases, the simulated protein structure data indicates a structure of a target protein. For example, simulated protein structure data may include computational representations or models of the three-dimensional structure and/or dynamics of a target protein. This data may be generated through various computational methods such as molecular dynamics simulations, Monte Carlo simulations, or other computational techniques that aim to predict or approximate the spatial arrangement, movements, and/or interactions of atoms and molecules within a protein structure over time. Simulated protein structure data may include information on atomic coordinates, bond lengths, angles, torsions, electrostatic interactions, and other physicochemical properties that describe the protein's conformation and/or behavior under simulated conditions.

Accessing the simulated protein structure data may include retrieving such data from a memory or other suitable data storage device or medium. Additionally or alternatively, accessing the simulated protein structure data may include simulating such data with the computer system, as described above.

One or more dynamics metrics are then generated based on the simulated protein structure data using the computer system, as indicated at step 04. The dynamics metric(s) may be generated for one or more residues in the protein. These dynamics metrics may provide information about the structural and functional properties of the protein. For instance, dynamics metrics may include quantitative measures or parameters that characterize the motion, flexibility, and interactions of a protein structure or its components over time. These metrics may be derived from simulated protein structure data or experimental measurements and may include, but are not limited to, measures of atomic fluctuations, correlations between residue movements, conformational changes, allosteric couplings, and/or other dynamic properties of the protein. In some cases, the dynamics metrics may include at least one of a dynamic flexibility index, a dynamic coupling index, and/or an asymmetric dynamic coupling index. In some instances, other metrics may also be calculated, including a solvent accessible surface area, one or more network features, or a number of contacts.

The dynamic flexibility index (DFI) may measure the degree of movement or flexibility of individual residues within the protein structure. This metric may help identify regions of the protein that are more mobile or rigid.

In general, the DFI metric calculates the relative flexibility/rigidity of individual residues in a protein. The DFI algorithm, which is developed using linear response theory and perturbation response scanning, calculates the average response of a residue as a result of a perturbation on every other residue in a protein. Taking advantage of the residue covariances, DFI provides position specific flexibility profiles.

$${[ Δ R ]}_{3 N \times 1} = {{[ H ]}_{3 N \times 3 N}^{- 1} [ F ]}_{3 N \times 1} \qquad Eq . 1$$

A Hessian matrix, H, is compiled from the second derivatives of potentials. The inverse of the Hessian matrix, H⁻¹, contains residue covariances. The covariance matrix can be generated from a protein structure by utilizing an elastic network model or gathered from a MD simulation of the protein, which implicitly accounts for amino-acid side chain interactions and solvent interactions. The latter can be used to calculate the dynamic metrics as a non-limiting example. The residue response vector, ΔR, in Eq. 1 is the resultant vector containing the magnitude of responses from multiplying the covariance matrix by the force vector, F. The DFI for position i, which computes the normalized fluctuation response of a position upon perturbation on the chain is calculated as:

$${DFI}_{i} = \frac{{\sum }_{j = n}^{N} {\vert Δ {R}^{j} \vert}_{i}}{{\sum }_{i = 1}^{N} {\sum }_{j = 1}^{N} {\vert Δ {R}^{j} \vert}_{i}} \qquad Eq . 2$$

$$where {\vert Δ {R}^{j} \vert}_{i} = \sqrt{〈 {( Δ R )}^{2} 〉}$$

is the magnitude of fluctuation response at position i due to a perturbation at position j.

The DFI score yields position specific information about the conformational dynamics of a protein system. Positions displaying low DFI scores are highly rigid. These sites often make more than an average number of interactions with their neighbors, which suggests that they represent crucial dynamic hubs in a protein. Conversely, positions with high DFI scores are often highly mobile regions of a protein. These sites do not contribute to the collective motion of a protein as substantially as the rigid regions.

The dynamic coupling index (DCI) may quantify the extent to which the motions of different residues are correlated, providing insights into how different parts of the protein may influence each other.

In general, DCI measures the allosteric coupling between residue pairs. To carry out DCI analysis of an enzyme or other protein structure, a random unit force may be applied to residues contained in structural features (e.g., loops or segments) of the protein and allowed to propagate through the protein until it reaches a residue distal from the initial perturbation location. After probing all active site residues, a magnitude of response to other residues in the protein may be calculated, which represents the strength of coupling between each active site residue and all other residues in the protein. A calculated DCI of position i suggests its response to a perturbation on position j and may be calculated as follows:

$${DCI}_{i j} = \frac{{\vert Δ {R}^{j} \vert}_{i}}{{\sum }_{j = 1}^{N} {\vert Δ {R}^{j} \vert}_{i} / N} \qquad Eq . 3$$

$$where {\vert Δ {R}^{j} \vert}_{i} = \sqrt{〈 {( Δ R )}^{2} 〉}$$

is the magnitude of fluctuation response at position i due to a perturbation at position j normalized over the average response of position i when any position in the protein is perturbed by a random Brownian force. Thus, DCIᵢⱼ>1 indicates that position i is more sensitive to perturbations occurring on position j. Alternatively, a position with a DCLᵢⱼ value lower than 1 is regarded as weakly coupled to the site j. Moreover, the dominance in dynamic control can be determined by calculating the asymmetry between residue locations i and j. DCIᵢⱼ is defined as the response of residue i when residue j is perturbed and DCIⱼᵢ represents the response of residue j when residue i is perturbed.

The asymmetric dynamic coupling index (DCI_{asym}) may capture directional relationships in the coupling between residues, potentially revealing hierarchical relationships in protein dynamics. DCI_{asym} of location i may be calculated as follows:

$${DCI}_{asym} = {DCI}_{ij} - {DCI}_{ji} \qquad Eq . 4$$

Given this definition, DCI_{asym} can take both positive and negative values. Accordingly, residues with DCI_{asym} values around zero (e.g., between −0.05 and +0.05, or within another suitable range from zero) may be considered to be dynamically coupled with protein structure features (e.g., loops, segments) in a symmetric fashion. The residues with DCI_{asym} values higher than the upper threshold (e.g., +0.05) are considered as “controlled” (e.g., loop controlled) and the ones with DCI_{asym} values lower than the lower threshold (e.g., −0.05) are considered as “controller” (e.g., loop controller).

In some cases, additional metrics may also be calculating, including a solvent accessible surface area (SASA) of the protein, one or more network features, and/or the number of contacts. The SASA metric may quantify the extent to which each residue is exposed to a surrounding solvent, which can be important for understanding protein-ligand interactions and protein function. In some cases, the SASA calculation may be employed by using the Naccess algorithm, which first creates a sphere with the radius of a water molecule and then rolls the sphere on the surface of the protein. The accessible surface area is calculated per residues by measuring the fraction of residue that is accessible to the solvent.

In some cases, network features such as betweenness, closeness, and eigenvector centrality, may also be computed. For instance, network analysis of protein structures webserver may be utilized to calculate betweenness, closeness, and eigenvector centrality. Betweenness measures how often an amino acid lies on the shortest path between two other amino acids in the protein. High-betweenness nodes have been previously shown as important residues for protein structure and function. These residues are relevant in proteins, as the shortest paths between nodes (i.e., distal sites and active sites) pass through these nodes. The closeness metric shows how easily an amino acid can be reached by other amino acids in the protein. Eigenvector centrality measures how well an amino acid is connected to other important amino acids in the protein. Amino acids that are more easily reached by others and well connected to other important amino acids are important for maintaining the overall stability and function of the protein.

To determine the average number of contacts, a molecular dynamics simulation trajectory can be analyzed by counting the Cα contacts within a certain distance (e.g., 10 Å) for each residue that appeared in over a threshold amount (e.g., 80%) of the frames in the trajectory sampled every 1 ns.

Protein characteristics data can then be generated by relating the dynamics metrics to functional behaviors of the protein using the computer system, as indicated at step 06. In some cases, patterns in the dynamics metrics may be analyzed and correlated with known or predicted functional properties of the protein. For example, regions with high flexibility may be associated with binding sites or catalytic regions, while strongly coupled residues may indicate important communication pathways within the protein.

The protein characteristics data can provide a detailed functional characterization of individual residues, protein domains, and the overall protein, linking structural dynamics to biological function. In some cases the protein characteristics data may include one or more flexibility and/or rigidity profiles of protein regions, indicating which areas are more mobile or stable. Additionally or alternatively, the protein characteristics data may include an identification of allosteric sites that may influence protein function from a distance, an identification of key residues involved in protein-protein interactions, and/or an identification of dynamic communication pathways within the protein structure. The protein characteristics data may also include a characterization of long-range dynamic couplings between different protein domains or residues, a characterization of conformational ensembles and major conformational states, and/or a characterization of intrinsically disordered regions and their functional roles.

In some other cases, the protein characteristics data may include a classification of residues or domains as “controllers” or “controlled” based on their dynamic influence. Additionally or alternatively, the protein characteristics data may include predictions of how mutations may impact protein activity and function, predictions of protein stability and folding/unfolding behaviors and/or predictions of how post-translational modifications may alter protein dynamics and function.

The protein characteristics data may also include insights into protein-ligand binding mechanisms and conformational changes and/or insights into enzyme catalysis mechanisms and rate-limiting steps. In still other cases, the protein characteristics data may include correlations between evolutionary conservation and dynamic properties of residues and/or quantification of entropy and free energy changes associated with protein motions.

Using the computer system, a report may be generated from the protein characteristics data, the functional behaviors, and/or the dynamics metrics, as indicated at step 08. The report may include textual information, quantitative information, data plots, images, models, or other textual, numerical, or visual representations of data that can be presented to a user via the computer system. The report may include a functional characterization of each residue in the plurality of residues and a functional characterization of the protein as a whole. The functional characterization may include indicating one or more protein residues or domains as “controller” regions and one or more protein residue domains as “controlled” regions. The report may further include identifying allosteric sites. The report may further indicate the predicted impact of mutating certain residues. This report may therefore provide a comprehensive analysis of the protein's structure-function relationships based on the dynamics data.

The functional characterization in the report may include various types of information. For instance, it may identify residues or regions that are particularly important for the function of the protein, such as identifying those residues involved in allosteric regulation or long-range communication within the protein. The report may also predict how mutations to specific residues might affect the function of the protein, based on their dynamic properties and relationships to other parts of the protein.

In some cases, the report may include visualizations or graphical representations of the dynamics data, making it easier for users to interpret the results. The report may also provide suggestions for further experimental studies based on the computational findings, helping to guide future assessment of the protein of interest.

In some cases, the method may include generating a report that indicates a prediction of an impact of one or more mutations to the protein based on the functional characterization of the protein and the dynamics metrics. The report may analyze how specific amino acid substitutions could affect the protein's structure, stability, or function. This analysis may be based on the quantified dynamics metrics and the functional behaviors determined for each residue and domain of the protein. In some cases, the report may categorize predicted mutations as potentially beneficial, neutral, or deleterious to protein function. The report may provide explanations for these predictions based on how the mutations could alter key dynamic properties or functional regions identified through the analysis.

In some implementations, the method may include generating a report that indicates a prediction of an impact of one or more drugs on the protein function based on the functional characterization of the protein and the dynamics metrics. This analysis may involve simulating the binding of drug molecules to the protein structure and evaluating how this interaction could affect the protein's dynamics and functional behaviors. The report may describe potential allosteric effects of drug binding, changes in flexibility or rigidity of specific protein regions, or alterations to long-range coupling dynamics between protein domains. In some cases, the report may predict whether a drug could enhance or inhibit the protein's function based on these simulated interactions and the previously determined functional characterization.

The method may utilize machine learning algorithms trained on known protein-drug interactions to improve the accuracy of these predictions. In some implementations, the report may rank multiple drug candidates based on their predicted impacts on protein function, providing a quantitative assessment to guide further experimental studies or drug development efforts. By combining the detailed functional characterization of the protein with predictions of mutation and drug impacts, the method may provide valuable insights for applications such as protein engineering, drug discovery, and understanding disease mechanisms related to protein dysfunction.

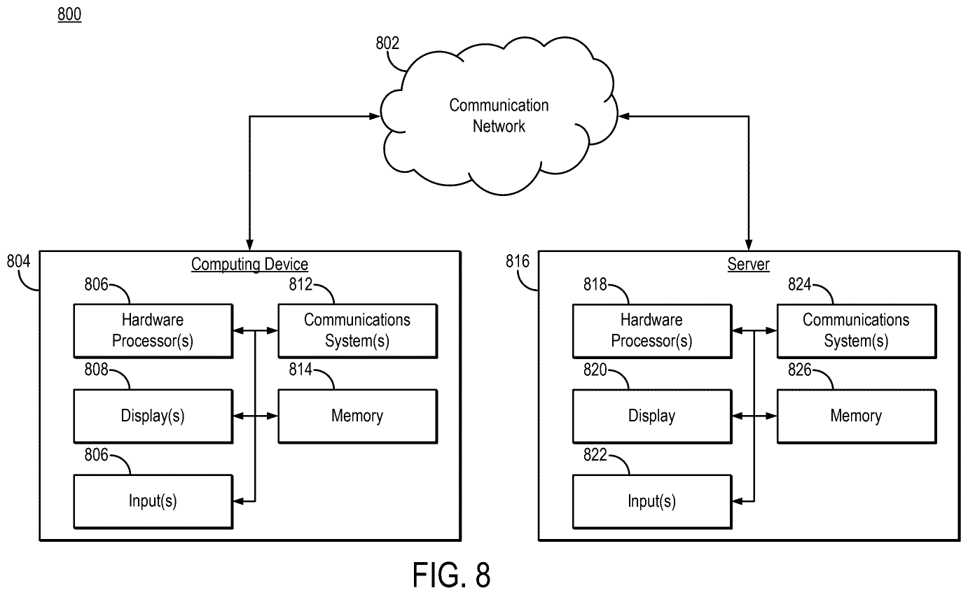

In FIG. 8, an example 800 of a system (e.g., a data processing system) for characterizing a protein in accordance with some embodiments of the disclosed subject matter is shown.

In some embodiments, computing device 804 and/or server 816 can be any suitable computing device or combination of devices, such as a desktop computer, a laptop computer, a smartphone, a tablet computer, a wearable computer, a server computer, a virtual machine being executed by a physical computing device, etc. As described herein, system 800 can present information about the characterized protein to a user (e.g., a researcher and/or a physician).

In some embodiments, communication network 802 can be any suitable communication network or combination of communication networks. In some embodiments, communication network 802 can be any suitable communication network or combination of communication networks. For example, communication network 802 can include a Wi-Fi network (which can include one or more wireless routers, one or more switches, etc.), a peer-to-peer network (e.g., a Bluetooth network), a cellular network (e.g., a 4G network, a 5G network, etc., complying with any suitable standard, such as CDMA, GSM, LTE, LTE Advanced, WiMAX, etc.), a wired network, etc. In some embodiments, communication network 802 can be a local area network, a wide area network, a public network (e.g., the Internet), a private or semi-private network (e.g., a corporate or university intranet), any other suitable type of network, or any suitable combination of networks. Communications links shown in FIG. 8 can each be any suitable communications link or combination of communications links, such as wired links, fiber optic links, Wi-Fi links, Bluetooth links, cellular links, etc.

FIG. 8 additionally shows an example of hardware that can be used to implement computing device 804 and server 816 in accordance with some embodiments of the disclosed subject matter. In some embodiments, computing device 804 can be used to execute one or more set of instructions to identify a behavioral catalog. In other embodiments, computing device 804 can be used to identify therapeutic interventions. In still other embodiments, computing device 804 can be used to identify a configuration of parameter of a gene regulatory network to perform a desired function.

As shown in FIG. 8, computing device 804 can include one or more hardware processor 806, one or more displays 808, one or more inputs 810, one or more communications 812, and/or memory 814. In some embodiments, processor 806 can be any suitable hardware processor or combination of processors, such as central processing unit, a graphics processing unit, etc. In some embodiments, display 808 can include any suitable display devices, such as a computer monitor, a touchscreen, a television, etc. In some embodiments, inputs 810 can include any suitable input device and/or sensors that can be used to receive user input, such as a keyboard, a mouse, a touchscreen, a microphone, etc.

In some embodiments, communication systems 812 can include any suitable hardware, firmware, and/or software for communicating information over communication network 802 and/or any other suitable communication networks. For example, communications systems 812 can include one or more transceivers, one or more communication chips and/or chip sets, etc. In a more particular example, communications systems 812 can include hardware, firmware and/or software that can be used to establish a Wi-Fi connection, a Bluetooth connection, a cellular connection, an Ethernet connection, etc.

In some embodiments, memory 814 can include any suitable storage device or devices that can be used to store instructions, values, etc., that can be used, for example, by processor 806 to present content using display 808, to communicate with server 816 via communications system(s) 812, etc.

Memory 814 can include any suitable volatile memory, non-volatile memory, storage, or any suitable combination thereof. For example, memory 814 can include RAM, ROM, EEPROM, one or more flash drives, one or more hard disks, one or more solid state drives, one or more optical drives, etc. In some embodiments, memory 814 can have encoded thereon a computer program for controlling operation of computing device 804. In such embodiments, processor 806 can execute at least a portion of the computer program to present content (e.g., images, user interfaces, graphics, tables, etc.), receive content from server 816, transmit information to server 816, etc.

In some embodiments, server 816 can include a processor 818, a display 820, one or more inputs 822, one or more communications systems 824, and/or memory 826. In some embodiments, processor 818 can be any suitable hardware processor or combination of processors, such as a central processing unit, a graphics processing unit, etc. In some embodiments, display 820 can include any suitable display devices, such as a computer monitor, a touchscreen, a television, etc. In some embodiments, inputs 822 can include any suitable input devices and/or sensors that can be used to receive user input, such as a keyboard, a mouse, a touchscreen, a microphone, etc.

In some embodiments, communications systems 824 can include any suitable hardware, firmware, and/or software for communicating information over communication network 802 and/or any other suitable communication networks. For example, communications systems 824 can include one or more transceivers, one or more communication chips and/or chip sets, etc. In a more particular example, communications systems 824 can include hardware, firmware and/or software that can be used to establish a Wi-Fi connection, a Bluetooth connection, a cellular connection, an Ethernet connection, etc.

In some embodiments, memory 826 can include any suitable storage device or devices that can be used to store instructions, values, etc., that can be used, for example, by processor 818 to present content using display 820, to communicate with one or more computing devices 804, etc. Memory 826 can include any suitable volatile memory, non-volatile memory, storage, or any suitable combination thereof. For example, memory 826 can include RAM, ROM, EEPROM, one or more flash drives, one or more hard disks, one or more solid state drives, one or more optical drives, etc. In some embodiments, memory 826 can have encoded thereon a server program for controlling operation of server 816. In such embodiments, processor 818 can execute at least a portion of the server program to transmit information and/or content (e.g., results of a tissue identification and/or classification, a user interface, etc.) to one or more computing devices 804, receive information and/or content from one or more computing devices 804, receive instructions from one or more devices (e.g., a personal computer, a laptop computer, a tablet computer, a smartphone, etc.), etc.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

### Example: Allosteric Regulatory Control in Dihydrofolate Reductase is Revealed by Dynamic Asymmetry

In an example study, the relationship between mutations and dynamics in Escherichia coli dihydrofolate reductase (DHFR) was investigated using the computational methods described in the present disclosure. The study focused on the M20 and FG loops, which are known to be functionally important and affected by mutations distal to the loops. Molecular dynamics simulations were used and position-specific metrics were developed, including the dynamic flexibility index (DFI) and dynamic coupling index (DCI), to analyze the dynamics of wild-type DHFR. The results were compared with existing deep mutational scanning data. The analysis showed a statistically significant association between DFI and mutational tolerance of the DHFR positions, indicating that DFI can predict functionally beneficial or detrimental substitutions. An asymmetric version of the DCI metric (DCI_{asym}) was also applied to DHFR, which indicated that certain distal residues control the dynamics of the M20 and FG loops, whereas others are controlled by them. Residues that are suggested to control the M20 and FG loops by the DCI_{asym} metric were evolutionarily nonconserved; mutations at these sites can enhance enzyme activity. On the other hand, residues controlled by the loops were mostly deleterious to function when mutated and were also evolutionary conserved. These results suggest that dynamics-based metrics can identify residues that explain the relationship between mutation and protein function or can be targeted to rationally engineer enzymes with enhanced activity.

Example 1 uses DHFR as an example protein. The approaches described in example 1 could also be applied to any other protein or enzyme.

The human-microbial antibiotic arms race has prompted extensive research efforts aimed at both developing new drugs and gaining a complete understanding of druggable enzymes. One such enzyme is DHFR, which has been investigated for its fundamental role in 5,6,7,8-tetrahydrofolate synthesis. Due to an abundance of biophysical data, DHFR from Escherichia coli represents an excellent model system for studying the relationship between protein dynamics and function. The catalytic activity of E. coli DHFR has been extensively studied. One major achievement in these studies was the crystallization of DHFR in conformations that represent intermediate steps of the enzymatic reaction pathway. These experiments revealed that multiple loops in DHFR are implicated in its function. For example, the M20 loop (residues 9-24) controls access to the active site and the FG loop (residues 116-132) stabilizes the M20 loop through hydrogen bonding interactions. Mutations on both of these loops have been reported to severely limit the activity of DHFR, whereas positions distal to these sites can be altered to enhance activity.

The dynamics of E. coli DHFR have also been thoroughly studied in an effort to gain insight into the impact of point mutations on its activity. These studies revealed that mutations in DHFR often modulate the enzyme's activity indirectly and at a distance. Namely, in a series of computational and experimental studies, mutations distal to the active site of DHFR were shown to alter hydrogen bonding interactions and the rotamers of residues close to the active site through a network of interacting residues. The role of dynamics in DHFR function has been studied previously, but a general relationship between dynamics of each position and their contribution to the activity has yet to be elucidated.

It was hypothesized in the example study that the position-specific dynamic features of DHFR can shed light on the diverse impact of mutations on its activity. Therefore, the dynamics of DHFR were examined utilizing three computational metrics: DFI, DCI, DCI_{asym}. The DFI metric measures the normalized magnitude of response of a residue to perturbations applied on all other amino acids; a high DFI value indicates high flexibility, conversely, a low DFI score suggests that a residue is highly rigid. The DCI metric reports on the dynamic coupling between residues. A high DCI value indicates high dynamic coupling between residues i and j, while a low DCI score implies weak coupling between these residues. Due to the complex conformational dynamics of a protein, the DCI score between two distal, noninteracting residues is not necessarily symmetric. The DCI asymmetry (DCI_{asym}) advantageously reports the difference in fluctuation response of residue i when j is perturbed versus the response of residue j when i is perturbed (DCIᵢⱼ−DCIⱼᵢ). DCI_{asym} can therefore be used to assess which of a pair of residues dominates the control of motion between them.

In this example study, DFI, DCI, and DCI_{asym} were applied to molecular dynamics (MD) simulations of DHFR. Because the M20 and FG loops of DHFR are highly important for its function, the DCI and DCI_{asym} metrics were used to assess whether the distal regions of DHFR dynamically modulate these loops. These analyses were then compared to a published deep mutational scanning dataset (Thompson et al., 2020), which allowed for linking the activity of DHFR to its dynamics. The dynamics metrics provided a link between previously reported mutational data and collective motions of the enzyme. In particular, DCI_{asym} allowed for classifying a given residue position as “controlled” (e.g., dynamically controlled by the loops) if its fluctuation response to a perturbation on M20 and FG loops was considerably lower than the response of M20 and FG loops when that residue was perturbed. If the opposite was true, that residue was classified as a “controller” (e.g., the residue is dynamically controlling the loop). When the mutational outcome of the controlled and controller positions was analyzed using deep sequencing data, it was observed that “controller” positions acted as allosteric hot spots (e.g., mutations at these positions modulate DHFR activity), whereas mutations on “controlled” positions were usually deleterious. Thus, dynamics based approaches (particularly the “controller” and “controlled” classification) can be used to better understand the relationship between protein dynamics and function in other proteins.

AMBER molecular dynamics software (Salomon-Ferrer et al., 2013) was used to study the dynamics of E. coli DHFR. The protein system was parametrized with ff14SB force field (Maier et al., 2015) and solvated with TIP3P explicit water model using minimum 16 Å distance from the protein to define the box size. The solvated protein was neutralized by sodium and chlorine ions and the energy was minimized with a steepest descent algorithm by 10,000 steps. The production trajectory was simulated with isothermal, isobaric, constant number of particles ensemble at 300 K and 1 bar pressure. Langevin thermostat was utilized to maintain the kinetic temperature of 300 K, and the pressure was regulated by the Berendsen barostat. Additionally, a SHAKE algorithm was used constrain the hydrogens. The simulation was run for 2 μs until convergence was achieved. The simulation converged when the root mean square deviation between the highest sampled conformation in consecutive time windows (i.e., the last 300 ns windows and the 300 ns window sequentially before it) was lower than 1 Å. Window sizes ranging from 100 ns up to 1 μs were used to determine convergence. Dynamics metrics (e.g., DFI, DCI, DCI_{asym}) were calculated from the simulated protein structure data using the techniques described above.

### Results

### Distinguishing Tolerant Versus Nontolerant Mutations and Understanding Mutational Outcomes Using Dynamic Flexibility Analyses

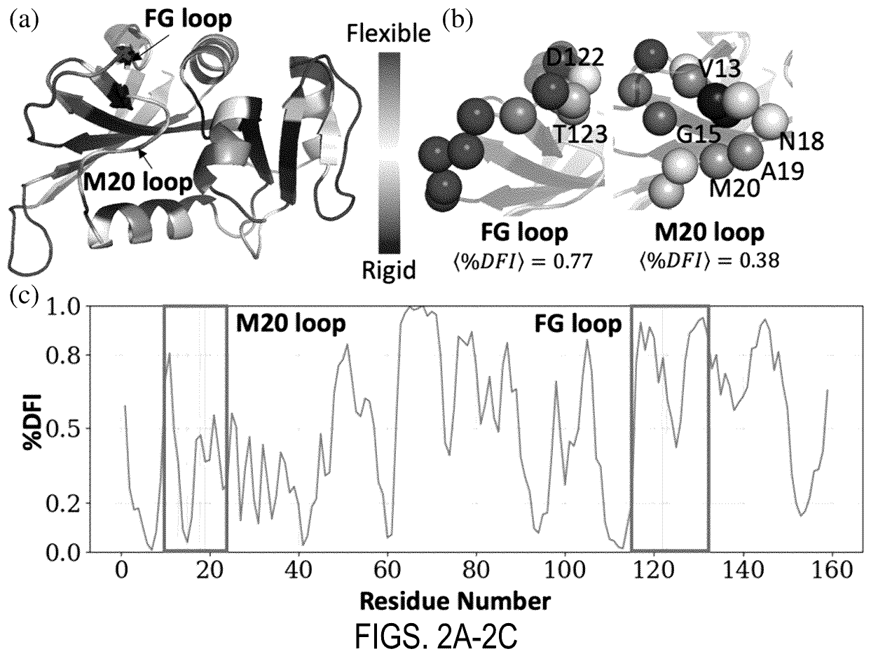

To gain insight into the impact of mutations on DHFR activity, DFI was first used to investigate the enzyme (FIGS. 2A-2C). In the DFI analysis, Brownian force perturbation was used to capture each position's response to random perturbations exerted on the protein chain. When a mutation creates a disturbance in the equilibrium dynamics of the protein, the local network of interactions surrounding the mutational site is often altered. Thus, the DFI value of a position can give a first order approximation of the impact that a mutation at that site might have on the enzyme's activity. It has been previously demonstrated that a high correlation exists between DFI values and modulation of protein function by disease-related mutations. Rigid locations identified by the DFI metric may be linked to disease related outcomes when mutated; alternatively, flexible locations are less prone to these types of disadvantageous mutations.

The example described above to study the relationship between dynamics and function in DHFR began with MD simulations of the enzyme using a model of apo DHFR (PDB ID: 1rx2) from the Protein Databank. The simulations were focused on the apo protein in this study because previous studies using NMR suggested that the apo enzyme also samples bound state dynamics. Thus, it was contemplated that use of an apo structure would provide insight into the dynamics of DHFR in conformations present in both the apo and bound forms. These MD simulations were then analyzed using the DFI metric, which revealed that previously known functionally important M20 and FG loops display dynamics profile different than each other (FIGS. 2A-2C). It was previously observed that residues that directly interact with ligands are often more rigid, and maintenance of this rigidity is important for enzyme function. A similar trend with residue M20 and its neighbors (N18 and A19) in the M20 loop (FIG. 2C), which directly interact with the substrate of DHFR, was observed in this study. Based on the analysis in the example study, these positions were more rigid than the remainder of residues in the loop. Moreover, residues D122 and T123 in the FG loop, and V13 and G15 on the M20 loop (FIG. 2C) were also found to be less flexible relative to other residues within these loops. These residues have previously been shown to stabilize the neighboring loops. In agreement with previous studies, substitutions at M20 and FG loop positions with low DFI scores were experimentally shown to drastically diminish (if not abolish) the activity of DHFR. Overall, the M20 loop showed a lower average DFI score ( [symbol] % DFI [symbol] =0.38) compared to the FG loop ( [symbol] % DFI [symbol] =0.77) implying modulation of DHFR activity by these loops are different (FIG. 2B).

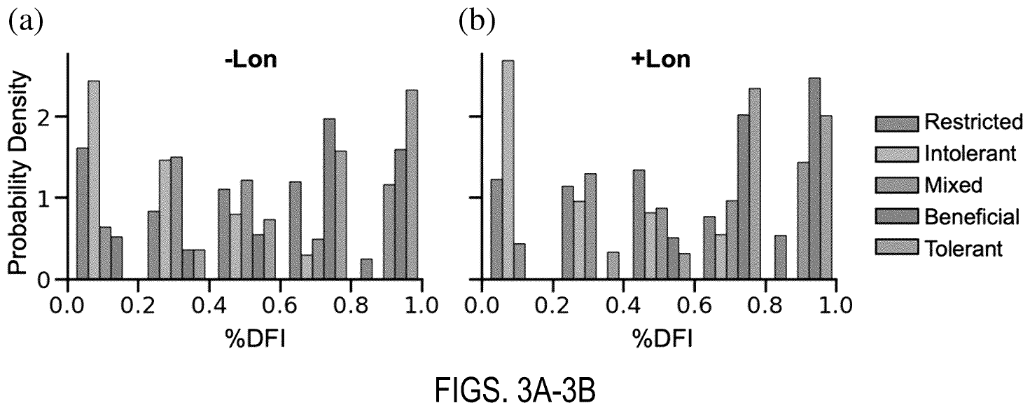

To further understand the implication of the conformational dynamics of residues related to function in DHFR, the data obtained using the DFI metric were related to previously reported experimental data (Thompson et al., 2020). Namely, a per residue functional classification, in which positions with advantageous mutations (named “Beneficial”), positions with WT-like behavior (named “Tolerant”), positions that possess both advantageous and disadvantageous mutations (named “Mixed”), residues with mostly disadvantageous mutations (named “Restricted”), and locations that exhibited almost no activity when mutated away from the wild-type amino acid (named “Intolerant”) were used in this analysis. When positions belonging to the aforementioned categories were analyzed from the perspective of their DFI values, the following trends were observed both in the absence (−Lon) and the presence (+Lon) of Lon protease (FIG. 3A): “Tolerant” and “Beneficial” mutations were more commonly found in residues with high % DFI scores, suggesting that more flexible residues are better able to accommodate mutations without negatively impacting protein function. In contrast, “Intolerant” and “Restricted” mutations were more commonly found in residues with lower % DFI scores, suggesting that more rigid residues are less able to tolerate mutations without negatively impacting protein function. The difference between “Tolerant” and “Intolerant” distributions are statistically significant (p=0.0002). As well as the difference in distributions of “Beneficial” and “Restricted” with a p value of 2e-05. The “Mixed” residues did not show a particular trend toward either rigid or flexible.

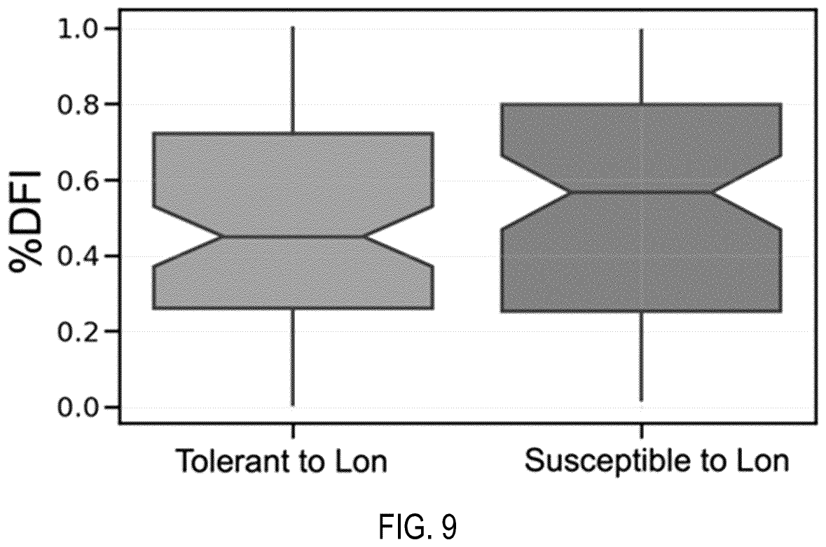

Moreover, a broad range of mutations, including many advantageous ones, face penalties due to Lon protease activity. To understand the relationship between DFI and protease sensitivity of residues, the distribution of DFI values was investigated. The analysis focused on two distinct categories: residues that exhibit tolerance to Lon (e.g., residues denoted as “Beneficial” when Lon is absent that remain “Beneficial” when Lon is present), and residues that are susceptible to Lon (e.g., those originally labeled as “Beneficial,” but which become “Restricted” in the presence of Lon) (FIG. 9). The box plots in FIG. 9 show that residues that are susceptible to the presence of Lon protease overall have a slightly higher DFI value compared to residues that are tolerant, suggesting that enhanced flexibility and low rigidity might play a role with stability. In summary, these results support the strength of the DFI metric in assessing the functional outcome of mutations on the protein, regardless of whether they are proximal to or distal from functionally important loops.

### Asymmetry in Dynamic Coupling Reveals Allosteric Mutational Sites

Mutations found far from (but dynamically coupled to) functionally important loops, which may be termed “allosteric mutations,” have previously been shown to substantially affect enzyme activity. The manner in which this long-range dynamic communication propagates through proteins can be highlighted with the DCI metric. A high DCI value indicates high dynamic coupling between residues i and j, suggesting that strong communication between these residues exists. A low DCI score implies weak coupling between residues and suggests the absence of strong communication between them.

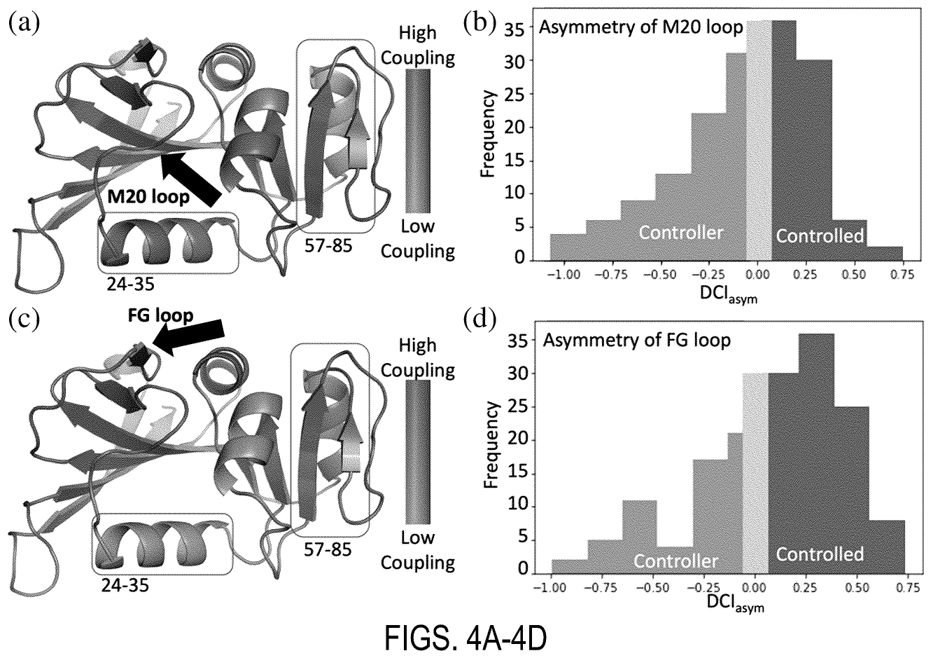

DCI analysis was applied to the M20 and FG loops to explore how these loops affect protein activity. Dynamic coupling analyses reveal that the M20 and FG loops, despite being close to each other, exhibit different long-distance interactions with the rest of the protein (FIG. 4A). It was also observed that each loop is dynamically coupled to specific regions within DHFR. For example, helix B, which spans residues 24-35, is more coupled to the M20 Loop (FIG. 4A), while the FG loop is highly coupled to β sheets C and D and the helical “E region” (residues 57-85, FIGS. 4A-4D).

The complex network of the protein suggested a disparity in dynamic coupling between positions that could be understood by an asymmetry in communication (FIGS. 4A-4D). Since each residue directly contacts a distinct set of neighboring residues, each position in a protein has a unique coupling network. Moreover, the dynamic coupling for position i with respect to j is not necessarily symmetric to the dynamic coupling of j to i. Thus, changes at position i may have larger effect on the flexibility of position j and vice versa. To capture this asymmetry, a novel metric DCI_{asym} was used, as described above. If the magnitude of difference between dynamic coupling scores of positions i to j (DCIⱼᵢ) versus the coupling of j to i (DCIᵢⱼ) is significant, an asymmetry in communication between the two residues will exist. This asymmetry in communication can be informative as to why certain amino acid substitutions at particular positions are more deleterious or beneficial to activity and vice versa.

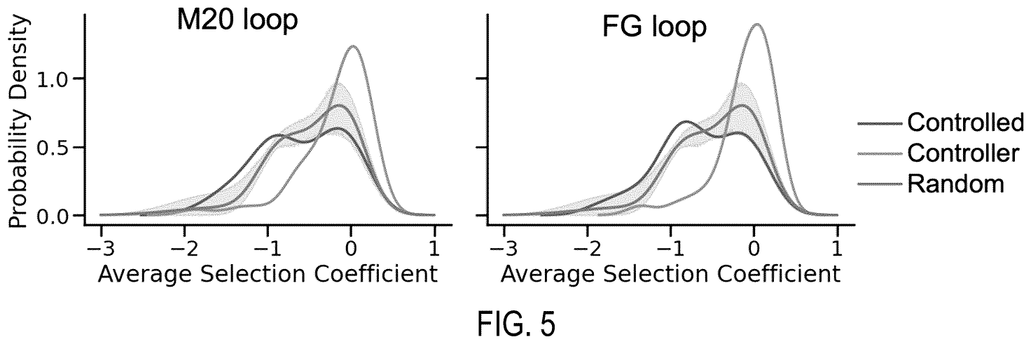

To explore how dynamic coupling between the M20 loop and the B helix or the FG loop and the E helix and the C and D sheets affected enzyme function, dynamic coupling was considered in both directions using the DCI_{asym} metric. DCI_{asym} was first applied to the M20 loop. DCI_{asym} values between −0.05 and 0.05 were considered to suggest that both positions are symmetric in their coupling; in other words, neither position has dominance. Alternatively, a residue would be defined as an “M20 controlled position” when its average DCI_{asym} score (calculated by taking the average of all M20 loop positions; i.e., [symbol] DCI_{asym} [symbol] ) was positive and larger than 0.05, and as an “M20 controller position” when [symbol] DCI_{asym} [symbol] was negative and lower than −0.05 (FIG. 4B). The same analysis was repeated on the FG loop (FIG. 4D). The “controlled”/“controller” categorization of residues was then compared with average selection coefficient values (FIG. 5). Selection coefficient values represent the impact of a mutation to DHFR activity relative to wild type. A mutation with a selection coefficient value around zero (±0.2) was considered as neutral. Values higher than 0.2 are beneficial to function, and conversely values lower than −0.2 are deleterious.

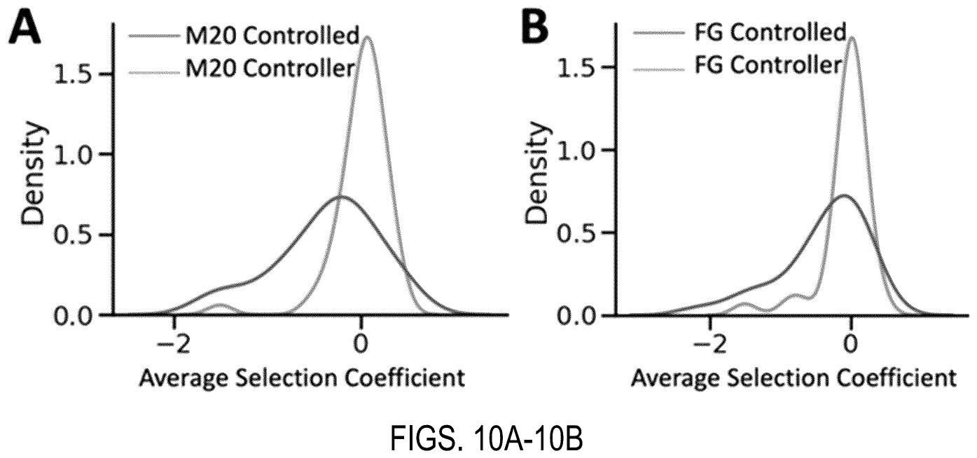

When investigated, the distribution of average selection coefficient values of “controlled” and “controller” residues displayed a different pattern (FIG. 5 and FIG. 10A). When residues that control the M20 loop were considered, the peak of the distribution was observed to be above zero, which indicates that mutations at these positions have, on average, a positive impact on the activity. Conversely, sites “controlled” by residues in the M20 loop displayed a broad distribution with high density around very negative values. This suggests that mutations at these residues have a deleterious effect on protein activity. A similar trend is observed when the FG loop is targeted with DCI and DCI_{asym} analyses (FIG. 5; FIG. 10B). Furthermore, comparison of the average selection coefficient distributions of “controlled” and “controller” residue positions with that of randomly selected positions revealed the statistical significance of the distribution of these classifications in distinguishing the impact of mutations on activity (FIG. 5).

### Beneficial Mutations are Enriched at Controller Sites

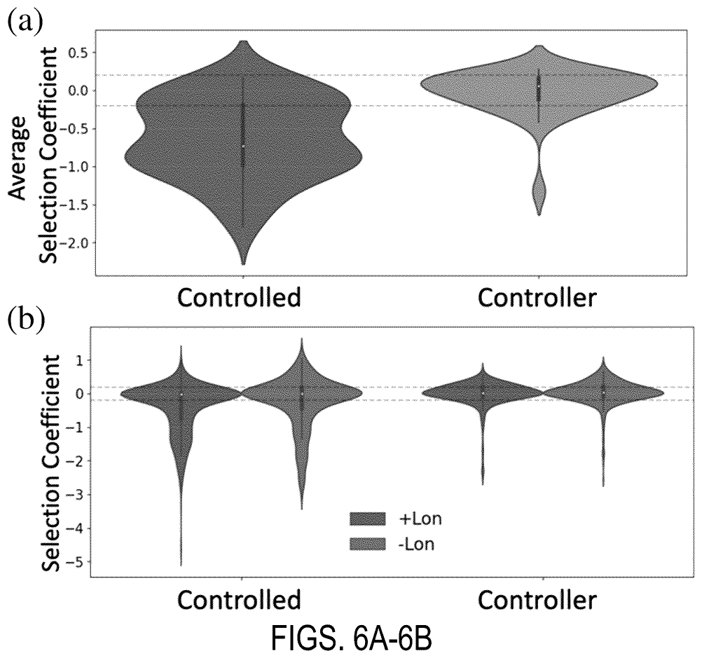

To further assess the impact of amino acid substitutions on residues with variety of control over the M20 and FG loops, the “controlled” and “controller” designations from each loop were combined. Namely, in this analysis, a residue was defined as a “controller” if it exerted control over both the M20 and FG loops simultaneously and was considered a “controlled” residue otherwise. The average selection coefficient value distributions of “controlled” and “controller” residues differed from each other when viewed in this way (FIG. 6A). Controller residues generally present more activity-enhancing amino acid substitutions compared to residues in the “controlled” category. The peak of the distribution of “controlled” residue mutations was below the neutral range (near −1.0). This indicated that mutations to “controlled” positions more often yield deleterious outcomes with respect to function. On the other hand, mutation of “controller” positions could gradually modulate function both positively and negatively and could therefore act like rheostatic switches. This suggests that the M20 and FG loops themselves are highly conserved due to functional constraints. However, those residues that control the loops can affect the overall enzyme function by distally altering functionally important residues.

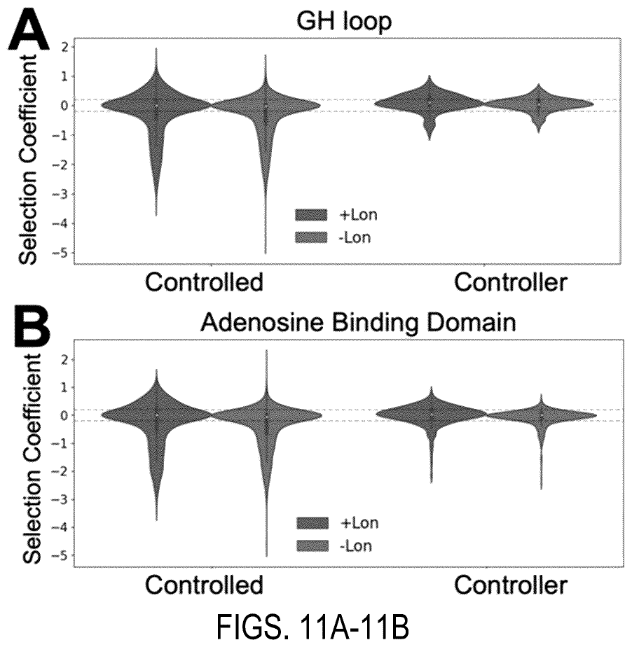

To remove any bias that arose from averaging, the distributions were also obtained using selection coefficient values for every mutation (as opposed to average values for all mutational outcomes per position). When all selection coefficient values were considered, the differences in asymmetry between “controlled” and “controller” residues was more pronounced (FIG. 6B). Additionally, when other functionally important sites, e.g., the GH loop (spanning residues 142-149) and the Adenosine Binding Domain (spanning residues 63, 64, and 65) were investigated, the results were similar to those found with M20 and FG loops (FIG. 11). This striking difference in the distribution of functional outcomes of mutations on “controlled” versus “controller” residues illustrates the importance of dynamic allosteric control. Previously, asymmetry in dynamic coupling was explored by analyzing 591 pathogenic missense variants in 144 human enzymes. It was shown that many mutations far from the active site exhibit deleterious behavior (sometimes leading to pathogenicity) due to their high coupling with the active site. Furthermore, it was also observed that these mutations are coupled to the active site, but the coupling strength (DCI score) of the mutation sites back to active site is not as high, showcasing an imbalance in coupling strength (asymmetry). The “controller” and “controlled” classification developed in the present disclosure highlights the importance of dynamic coupling to active sites. In addition, it highlights the degree to which asymmetry in this coupling can modulate function in a positive or negative direction.

### Leveraging Asymmetry in Dynamic Coupling for Fine-Tuning Function: a Comparative Analysis of Other Metrics and Functional Outcomes

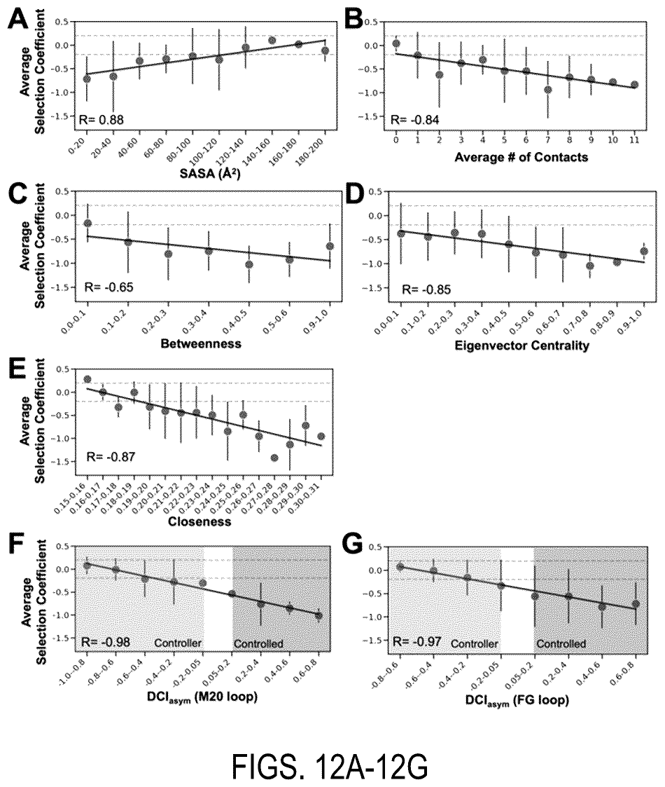

To evaluate the effectiveness of the DCI_{asym} metric in identifying residue positions with diverse activities upon mutations, it was compared with several other metrics that are commonly used to identify functionally critical sites, such as solvent-accessible surface area (SASA), average number of contacts, as well as network metrics including betweenness, closeness, and eigenvector centrality values (FIGS. 12A-12G). After computing these metrics for each residue, positions sharing similar values were grouped using histograms and the average and variance of the experimental average selection coefficients of the positions residing in each bin/group were analyzed.

The average of SASA values in each bin correlated with average experimental values (R=0.88), indicating that highly accessible residues were more likely to have neutral outcomes upon mutation. The average number (#) of contacts showed a correlation of −0.84 with experimental fitness, but the deviation at medium ranges suggested that, on average, most of these residues were deleterious to function when mutated. The betweenness scores showed a relatively low correlation with the experimental values (R=−0.65). Despite its strong correlation with fitness (R=−0.85), the observed average negative fitness values with all eigenvector centrality ranges suggested that underlying factors beyond altered functional outcomes upon substitution were not fully captured by this metric. The closeness measure identified residue positions with high experimental fitness scores (>0.2) but failed largely to identify residues with deleterious behavior.

In contrast, when dynamic-based metrics (e.g., DCI_{asym}) of the M20 and FG loops were analyzed in the same manner, the results showed that the DCI_{asym} metric was the most effective in capturing the trend of changing fitness for both the M20 loop and FG loop, with high correlations of −0.98 and −0.97, respectively. This indicated that as a position becomes more controlled, mutations at that site are more deleterious; alternatively, “controller” residues yield more neutral or beneficial outcomes when mutated. These findings suggest that the “controlled” and “controller” classification based on the DCI_{asym} metric can not only provide high accuracy in identifying and characterizing residues, but can also help identify “controller” sites that may subtly tune the enzyme's function when mutated.

### Examining the Interplay of Asymmetry in Dynamic Coupling and Evolutionary Conservation

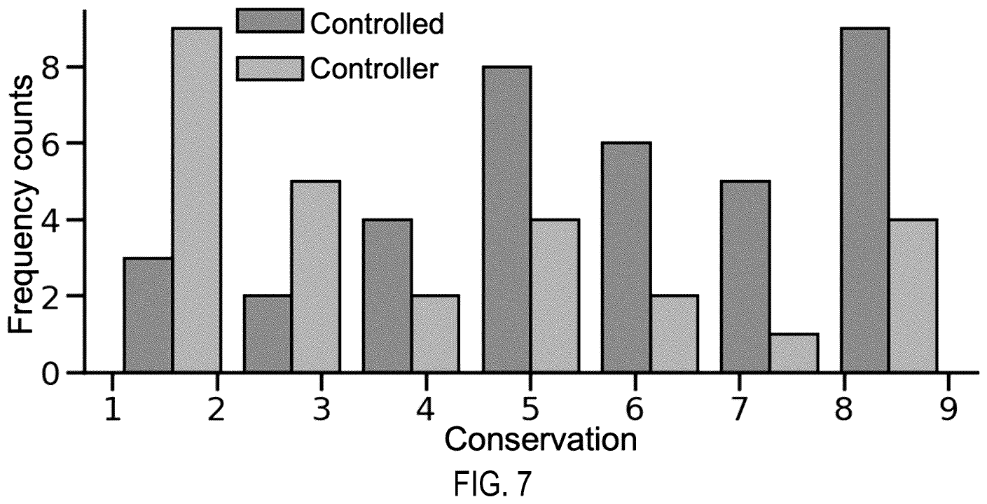

To gain insight into how “controller”/“controlled” categorization relates to a position's conservation, the ConSurf database (Ben Chorin et al., 2020; Goldenberg et al., 2009) was used to evaluate conservation of each site. Namely, the distribution of conservation of each residue in DHFR was considered with respect to its asymmetry categorization (FIG. 7). Previous studies have shown that the positions of the M20 and FG loops are structurally and evolutionarily conserved. When the conservation of residues controlled by the M20 and FG loops was investigated, the “controlled” residues were found to be highly conserved. This suggests that mutations of “controlled” residues often yield deleterious outcomes and are therefore filtered out by natural selection. Alternatively, “controller” sites are found to be nonconserved, indicating that they can accommodate a diverse number of mutations. This behavior was observed in the deep mutational scanning data where mutations on “controller” residues enable enhancement or modular changes in the activity of DHFR. Conservation analyses showed that “controlled” residues are highly conserved, while “controller” sites are nonconserved, allowing for a diverse number of mutations and enabling enhancement or modular changes in the activity of DHFR. Indeed, these results agree with the deep sequencing data where the mutations on controlled sites are usually deleterious; therefore, they are also eliminated during evolution.

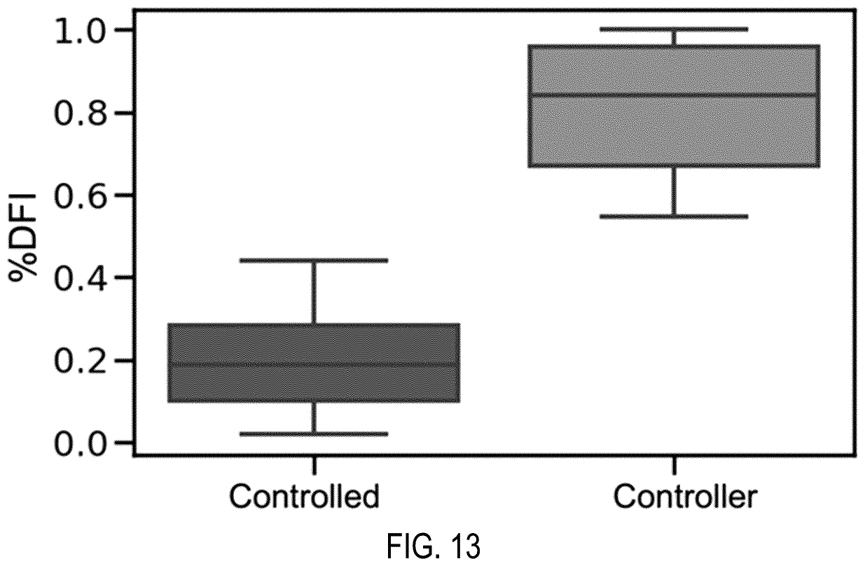

To gain a deeper insight into why a distinction between “controlled” and “controller” residue conservation exists, the flexibility of these positions was examined. The analyses demonstrate that “controlled” residues are often highly rigid with an average % DFI score of 0.2 (FIG. 13). Mutations occurring at these rigid sites typically have detrimental effects on function. In contrast, the “controller” residues exhibit a higher average % DFI value of 0.8 (FIG. 13), indicating high flexibility. This flexibility enables these positions to tolerate a broader range of amino acid changes. Consequently, selectively targeting “controller” residues holds promise for random mutagenesis or rational design approaches aiming to finely adjust the activity of DHFR. We believe our dynamics metrics DFI and DCI_{asym} could uncover these positions in other proteins as well.

### Conclusion

In this study, it was shown that dynamics-based metrics can be utilized to better understand the functional outcomes of mutations in DHFR. DFI scores displayed great promise in differentiating positions that might lead to beneficial or deleterious functional changes. The DCI metric revealed that the long-distance dynamic coupling between the M20 loop and other residues in DHFR differs significantly differs from that of FG loop. These diverse allosteric features are further investigated with our novel DCI_{asym} metric. The observed differences between residues that are controlled by or control two important loops in DHFR highlight how mutation of “controller” residues can fine tune enzyme activity through dynamic allostery. In addition, the evaluation of evolutionary conservation of “controlled” versus “controller” positions indicated that the “controller” sites are more amenable to mutations. On the other hand, “controlled” sites were more conserved since mutations to these sites often results in loss of function. Although this example study was carried out using DHFR, the conclusions drawn in this work display great promise for using dynamics metrics to gain a better understanding of how residues distal from functional portions of proteins can potentially modulate protein activity without compromising the fold.

The present disclosure has described one or more preferred embodiments, and it should be appreciated that many equivalents, alternatives, variations, and modifications, aside from those expressly stated, are possible and within the scope of the disclosure.

1. A method of characterizing an allosteric site of a protein, the method comprising:
(a) accessing simulated protein structure data with a computer system, wherein the simulated protein structure data indicate a structure of the protein;
(b) quantifying, using the computer system and based on the simulated protein structure data, a plurality of dynamics metrics for a plurality of residues in the protein;
(c) relating the plurality of dynamics metrics to functional behaviors of the protein using the computer system; and
(d) generating a report from the functional behaviors and the dynamics metrics using the computer system, wherein the report comprises a functional characterization of each residue in the plurality of residues and a functional characterization of the allosteric site of the protein.

2. The method of claim 1, wherein the protein structure data is simulated using molecular dynamics.

3. The method of claim 1, wherein the plurality of dynamics metrics comprises at least one of a dynamic flexibility index, a dynamic coupling index, or an asymmetric dynamic coupling index.

4. The method of claim 1, wherein the plurality of dynamics metrics comprises a solvent accessible surface area.

5. The method of claim 1, wherein the protein comprises a drug target.

6. The method of claim 5, wherein the drug target comprises dihydrofolate reductase (DHFR).

7. The method of claim 1, wherein the functional characterization of the protein comprises indicating long-range coupling dynamics.

8. The method of claim 7, wherein the long-range coupling dynamics includes identifying controller residues or regions and controlled residues or regions.

9. The method of claim 1, wherein the report further comprises a functional characterization of one or more domains in the protein.

10. The method of claim 1, wherein the report indicates a prediction of an impact of one or more mutations to the protein based on the functional characterization of the protein and the dynamics metrics.

11. The method of claim 1, wherein the report indicates a prediction of an impact of one or more drugs on the protein function based on the functional characterization of the protein and the dynamics metrics.

12. A method of identifying allosteric sites in a protein, comprising:
simulating protein structure data using a computer system, wherein the protein structure data indicate a structure of the protein;
calculating an asymmetric dynamic coupling index (DCI_{asym}) for a plurality of residues in the protein using the computer system;
classifying residues as controller or controlled based on their DCI_{asym} values;
identifying allosteric sites in the protein based on the classification of residues as controller or controlled; and
generating a report that indicates the allosteric sites in the protein.

13. The method of claim 12, wherein classifying residues as controller or controlled comprises:
classifying residues with DCI_{asym} values above an upper threshold as controlled residues; and
classifying residues with DCI_{asym} values below a lower threshold as controller residues.

14. The method of claim 13, wherein the upper threshold is 0.05 and the lower threshold is −0.05.

15. The method of claim 12, further comprising calculating a dynamic flexibility index (DFI) for each residue in the protein.

16. The method of claim 15, wherein identifying the allosteric sites comprises identifying controller residues with high DFI values as potential allosteric sites.

17. The method of claim 12, wherein calculating the DCI_{asym} for each of the plurality of resides comprises:
calculating a dynamic coupling index (DCIᵢⱼ) for each of the plurality of residues, wherein the DCI of a residue position i indicates its response to a perturbation on another reside position j; and
calculating the DCI_{asym} values as a magnitude of difference between dynamic coupling scores of residue positions i to j (DCIᵢⱼ) versus dynamic coupling scores of residue positions j to i (DCIⱼᵢ).

18. The method of claim 12, further comprising predicting an impact of mutations on protein function at the identified allosteric sites.

19. The method of claim 18, wherein predicting the impact of mutations comprises classifying potential mutations as beneficial, neutral, or deleterious to protein function based on at least the DCI_{asym} values.

20. The method of claim 19, wherein the generated report further indicates a functional characterization of the identified allosteric sites and predictions for the impact of mutations at those sites.
